(19) United States
(12) Patent Application Publication
(10) Pub. No.: US 2017/0319243 A1
(43) Pub. Date: Nov. 9, 2017

(54) SPACER DEVICE FOR FIXING BAND BETWEEN SPINOUS PROCESSES

(71) Applicants: Hyeun-Sung Kim, Daejeon (KR); SOLCO BIOMEDICAL CO., LTD., Pyeongtaek-si (KR)

(72) Inventors: Hyeun-Sung Kim, Daejeon (KR); Hong-Won Yoon, Gunpo-si (KR)

(21) Appl. No.: 15/527,748

(22) PCT Filed: Nov. 30, 2015
(86) PCT No.: PCT/KR2015/012912

§ 371 Date: May 18, 2017

(30) Foreign Application Priority Data
Dec. 3, 2014 (KR) 10-2014-0172561

(51) Int. Cl. A61B 17/70; A61B 17/68; A61B 17/82

(52) U.S. Cl. CPC A61B 17/7067; A61B 17/70; A61B 17/68; A61B 17/82; A61B 17/7061

(57) ABSTRACT

The present invention relates to a spacer device for fixing a band between spinous processes, the spacer device comprising: a band unit which has a certain degree of tension and is disposed between a spinous process and an adjacent spinous process which protrude from the vertebrae, on the outer circumference of the spinous process and on the outer circumference of the adjacent spinous process so as to mutually fix the spinous process and the adjacent spinous process; and a spacer unit which is coupled to the band unit and is disposed in a space between the spinous process and the adjacent spinous process so as to fix the end of the band unit, wherein the spacer unit moves along the band unit from the outside of the body to the inside of the body in a state where the band unit penetrates the spacer unit, and thus the spacer unit is inserted into the space between the spinous process and the adjacent spinous process. Thereby, the spacer device can: efficiently and reliably fix the end of a band for mutually fixing adjacent spinous processes, while permitting fine motion during surgery for treatment of spinal diseases; acquire optimal procedure results and efficiently carry out surgical procedures regardless of skill due to a relatively inexpensive and simple configuration; take action in accordance with the body types of various patients; and provide a patient with a state identical to that of original vertebrae since a firmly fixed state can be maintained and, simultaneously, fine motion is permitted following a procedure.

### BACKGROUND OF THE INVENTION

### Field of the Invention

The present invention relates to a spacer device for fixing a band between spinous processes and, more particularly, to a spacer device for fixing a band between spinous processes, for fixing the end of a band which fixes adjacent spinous processes, but permits a minute movement upon an operation for treating a vertebral disease, such as a spinal canal stenosis or a degenerative disc disease.

### Discussion of the Related Art

A spinal canal is a passage through which a vertebral nerve passes. Holes at the rear of respective vertebrae are connected up and down to form a tunnel, and nerves continue from the brain to an arm and a leg through the tunnel.

If such a passage through which the vertebral nerve passes is narrowed, a patient is exposed to a disease, such as a spinal canal stenosis in which the patient feels a severe pain because the nerve is pressed.

The most common disc disease may be said to be a degenerative spinal canal stenosis.

Such a degenerative spinal canal stenosis is generated because as a vertebra experiences an aging process, a disc (intervertebral disc) that has caused a degenerative change pops out at the front and presses a nerve, a ligamentum flavum that surrounds the nerve becomes thick and hard-set at the back and presses the nerve, a facet joint located at the back of a spinal canal is swollen, and a vertebral nerve is pressed by a bone (bony spur) abnormally grown by the friction of the vertebra.

Recently, in order to treat such a spinal canal stenosis, an implant inserted between adjacent spinous processes is widely used.

In general, a vertebral implant is for the fusion of spinous processes of patients who suffer from a degenerative disc disease, a spondylolisthesis, an external injury and a tumor.

However, a conventional vertebral implant has problems in that a spinous process is damaged and the vertebral implant escapes between spinous processes because upper and lower spinous processes are not agglutinated so that they are moved separately, not together, caused by a long-time movement of vertebra.

Furthermore, there are problems in that since a ligament must be cut in the rear of the back and the vertebral implant must be inserted in order to insert the vertebral implant between the spinous processes, it takes long time to both operate and recover for a patient from the operation.

Furthermore, there is a problem in that the vertebral implant is not coupled to the upper and lower spinous processes perfectly and stably because a wing portion coupled to the upper and lower spinous processes is not configured to be adjusted according to the thickness of the spinous process after the vertebral implant is inserted between the spinous processes.

Furthermore, such a vertebral implant had a problem in that it is difficult to perform a procedure because the angle or width of a spinous process and adjacent spinous process is various depending on the body type of a patient.

In particular, such conventional vertebral implants had problems in that a production cost according to a mold design is high and the vertebral implants are economically inefficient because they have a complicate structure.

Furthermore, the conventional vertebral implants had a problem in that a high skill is required upon a procedure because the vertebral implants are complicated in structure as described above.

### PRIOR ART DOCUMENT

### Patent Document

Korean Patent No. 10-1004937

Korean Patent No. 10-0138068

Korean Patent No. 10-1333657

### SUMMARY OF THE INVENTION

The present invention has been invented to improve such problems, and an object of the present invention is to provide a spacer device for fixing a band between spinous processes, which efficiently and surely fixes the end of a band for fixing adjacent spinous processes, but permitting a minute movement upon an operation for treating a vertebral disease.

Furthermore, an object of the present invention is to provide a spacer device for fixing a band between spinous processes, wherein a procedure can be efficiently performed regardless of a skill and optimal procedure results can be obtained through a relatively cheap and simple configuration.

Furthermore, an object of the present invention is to provide a spacer device for fixing a band between spinous processes, which can be handled in accordance with various body types of patients and can provide a patient with the same state as that of the original vertebra by permitting a minute movement while maintaining a fast fixing state after a procedure.

In order to accomplish the objects, the present invention may provide a spacer device for fixing a band between spinous processes, including a band unit having a specific degree of tension, being disposed between a spinous process and adjacent spinous process protruded from vertebras and in the outskirts of the spinous process and the outskirts of the adjacent spinous process, and fixing the spinous process and the adjacent spinous process; and a spacer unit coupled to the band unit, disposed in a space between the spinous process and the adjacent spinous process, and having the end of the band unit, wherein the spacer unit moves from the outside of a body to the inside of the body along the band unit in the state in which the spacer unit has penetrated the band unit and is inserted into the space between the spinous process and the adjacent spinous process.

In this case, the spacer unit includes a spacer body through which a main band belonging to the band unit and disposed between the spinous process and the adjacent spinous process penetrates and a tightner which is coupled to the spacer body and through which the main band penetrates. An assistant band coupled to the main band penetrates the tightner via the outskirts of the spinous process. The other end of the main band penetrates the tightner via the outskirts of the adjacent spinous process. Each of the main band and the assistant band is fixed through the tightner.

In this case, the main band and the assistant band penetrate the tightner in the state in which the tightner has been coupled to the spacer body.

Furthermore, the spacer unit further includes a locker which is coupled to the tightner and which fixes the ends of the main band and the assistant band.

Furthermore, the spacer unit further includes a clamp which is fixed to the outside surface of the tightner and which finishes the ends of the main band and the assistant band fixed through the tightner.

Furthermore, the band unit includes a main band having a first length and permitting elastic transform and an assistant band having a second length shorter than the first length, permitting elastic transform, and having one end coupled at a specific point of the main band. The other end of the assistant band is disposed in the outskirts of the spinous process. One end of the main band is disposed in the outskirts of the adjacent spinous process. The other end of the main band is disposed after passing through the space between the spinous process and the adjacent spinous process. The other end of the main band penetrates the spacer unit.

Furthermore, the spacer body includes a main body of a barrel shape having one side open, a first screw thread depressed and formed from the other end of the main body toward one end and screwed onto the tightner, and a band slot penetrated from one end of the main body to the other end on one side of the main body so that the other end of the main band is inserted into the band slot from one end side of the main body and the other end of the main band is exposed from the other end side of the main body.

Furthermore, the tightner includes a first fastening part screwed onto the spacer body, a second screw thread being formed in an outer surface of the fast fastening part, a penetration part provided at the end of the first fastening part, wherein a first communication slot through which the other end of the main band penetrating the spacer body penetrates, a second communication slot through which the end of the assistant band penetrates, and a third communication slot through which one end of the main band penetrates penetrate the penetration part in the direction in which the first to third communication slots are fastened to the spacer body, the first communication slot, the second communication slot, and the third communication slot being disposed in the penetration part at equal intervals, and a second fastening part depressed from the end of the penetration part toward the first fastening part, a third screw thread being formed in an inner surface of the second fastening part.

Furthermore, the spacer device further includes a locker which is screwed onto the third screw thread of the second fastening part formed in the penetration part and which fixes the other end and one end of the main band and the end of the assistant band. The first communication slot, the second communication slot, and the third communication slot communicate with the inside of the penetration part.

Furthermore, the locker includes a third fastening unit which has a fourth screw thread screwed onto the third screw thread formed in an outer surface of the third fastening unit and a fixing body which is provided at the end of the third fastening unit, stepwise formed from the end of the penetration part toward the second fastening part, and seated and fixed to a receiving groove communicating with the first communication slot, the second communication slot, and the third communication slot and which fixes the main band and the assistant band. The main band and the assistant band are fixed between an inner surface of the receiving groove and an outer surface of the fixing body.

### Effects of the Invention

According to the present invention having the configuration, the following advantages can be accomplished.

First, in the present invention, upon a procedure for treating a vertebral disease, the end of the band unit which fixes adjacent spinous processes, but permits a minute movement can be fixed by the spacer unit efficiently and surely.

Furthermore, the present invention has a relatively cheap and simple configuration including the spacer body, the tightner, the locker, etc. along with the band unit. Accordingly, a procedure can be efficiently performed regardless of a skill and optimal procedure results can be provided to a patient.

Furthermore, according to the present invention, a minimal invasive surgery (MIS) is made possible through minimum incision because the spacer unit moves from the outside of the body to the inside of the body along the band unit and is inserted between two spinous process in the state in which the spacer unit has penetrated the band unit exposed to the outside of the body of a patient.

Furthermore, according to the present invention, an action can be taken in accordance with various body types of patients because the band unit is closely attached and fixed to a spinous process and an adjacent spinous process while permitting a gap between the spinous processes to some extent. A fast fixing state can be maintained and a minute movement is also permitted after a procedure because the state in which the spacer unit fixes the end of the band unit is maintained. Accordingly, the same state as that of the original vertebra will be provided to a patient.

### BRIEF DESCRIPTION OF THE DRAWINGS

FIG. 1 is a perspective view showing the state in which a procedure is performed on a spacer device for fixing a band between spinous processes according to an embodiment of the present invention between the spinous process and adjacent spinous process of a patient.

FIG. 2 is an exploded perspective view showing a coupling relation between the spacer body, tightner, and locker of a spacer unit, that is, a principal part of the spacer device for fixing a band between spinous processes according to an embodiment of the present invention.

FIG. 3 is a perspective view showing the state in which the spacer body, tightner, and locker of the spacer unit, that is, a principal part of the spacer device for fixing a band between spinous processes according to an embodiment of the present invention, have been coupled.

FIG. 4 is an exploded perspective view showing a relation in which a clamp is additionally coupled in the state in which the spacer body, tightner, and locker of the spacer unit, that is, a principal part of the spacer device for fixing a band between spinous processes according to another embodiment of the present invention, have been coupled.

FIG. 5 is a perspective view showing the state in which a clamp has been additionally coupled in the state in which the spacer body, tightner, and locker of the spacer unit, that is, a principal part of the spacer device for fixing a band between spinous processes according to another embodiment of the present invention, have been coupled.

FIG. 6 is a perspective view showing an overall structure of the spacer body of the spacer unit, that is, a principal part of the spacer device for fixing a band between spinous processes according to an embodiment of the present invention.

FIG. 7 is a perspective view showing an overall structure of the tightner of the spacer unit, that is, a principal part of the spacer device for fixing a band between spinous processes according to an embodiment of the present invention.

FIG. 8 is a perspective view showing an overall structure of the locker of the spacer unit, that is, a principal part of the spacer device for fixing a band between spinous processes according to an embodiment of the present invention.

FIGS. 9 to 11 are perspective views showing a process for sequentially coupling the spacer unit to the band unit using the spacer device for fixing a band between spinous processes according to an embodiment of the present invention and performing a procedure.

### DETAILED DESCRIPTION OF THE EMBODIMENTS

The merits and characteristics of the present invention and methods for achieving the merits and characteristics will become evident from embodiments described in detail later in conjunction with the accompanying drawings.

However, the present invention is not limited to the disclosed embodiments, but may be implemented in various different ways.

In the specification, the embodiments are provided to only complete the disclosure of the present invention and to allow a person having ordinary skill in the art to which the present invention pertains to completely understand the category of the invention.

Furthermore, the present invention is only defined by the category of the claims.

Accordingly, in some embodiments, well-known elements, well-known operations, and well-known technologies are not described in detail in order to avoid the present invention from being ambiguously construed.

Furthermore, in the entire specification, the same reference numerals denote the same elements, and terms used (mentioned) in the specification are for describing the embodiments and are not intended to limit the present invention.

In the specification, the singular form, unless specially described otherwise in the context, may include the plural form. An element and operation described as being “comprise (or include)” do not exclude the existence or addition of one or more elements and operations.

Unless defined otherwise, all of terms (including technological and scientific terms) used in the specification will be used as meanings which can be understood by a person having ordinary skill in the art to which the present invention pertains in common.

Furthermore, terms that are generally used and defined in dictionaries should not be construed as having ideal or excessively formal meanings unless defined otherwise.

Hereinafter, preferred embodiments of the present invention are described with reference to the accompanying drawings.

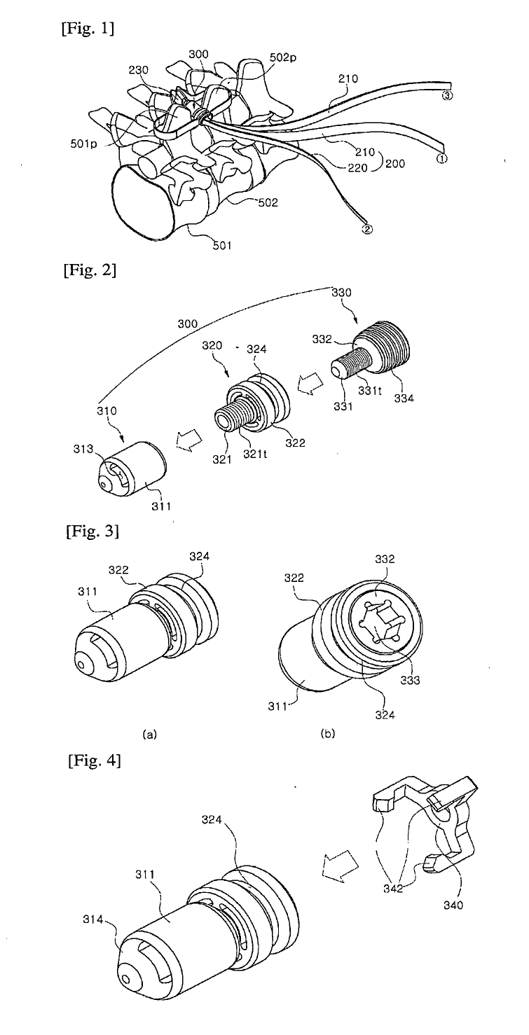

FIG. 1 is a perspective view showing the state in which a procedure is performed on a spacer device for fixing a band between spinous processes according to an embodiment of the present invention between the spinous process and adjacent spinous process of a patient.

Furthermore, FIG. 2 is an exploded perspective view showing a coupling relation between the spacer body, tightner, and locker of a spacer unit, that is, a principal part of the spacer device for fixing a band between spinous processes according to an embodiment of the present invention.

Furthermore, FIG. 3 is a perspective view showing the state in which the spacer body, tightner, and locker of the spacer unit, that is, a principal part of the spacer device for fixing a band between spinous processes according to an embodiment of the present invention, have been coupled.

Furthermore, FIG. 4 is an exploded perspective view showing a relation in which a clamp is additionally coupled in the state in which the spacer body, tightner, and locker of the spacer unit, that is, a principal part of the spacer device for fixing a band between spinous processes according to another embodiment of the present invention, have been coupled.

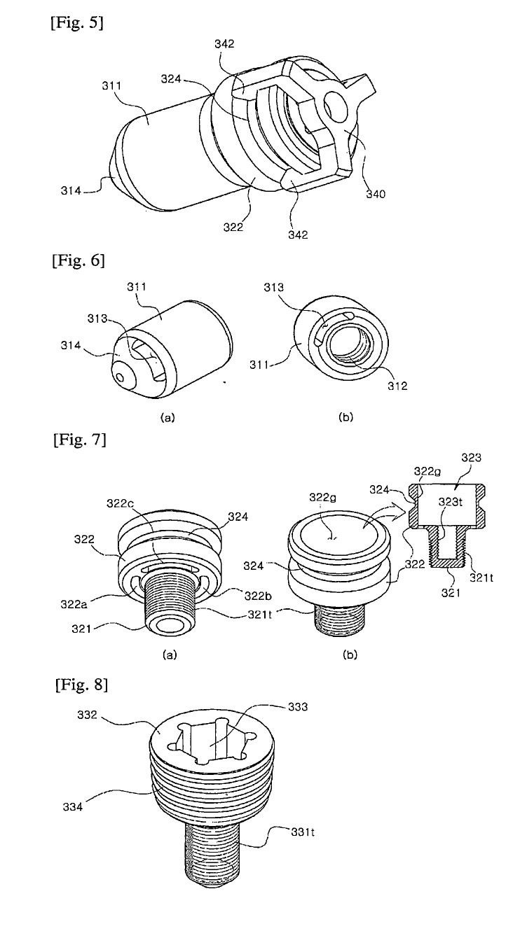

Furthermore, FIG. 5 is a perspective view showing the state in which a clamp has been additionally coupled in the state in which the spacer body, tightner, and locker of the spacer unit, that is, a principal part of the spacer device for fixing a band between spinous processes according to another embodiment of the present invention, have been coupled.

Furthermore, FIG. 6 is a perspective view showing an overall structure of the spacer body of the spacer unit, that is, a principal part of the spacer device for fixing a band between spinous processes according to an embodiment of the present invention.
Furthermore, FIG. 7 is a perspective view showing an overall structure of the tightner of the spacer unit, that is, a principal part of the spacer device for fixing a band between spinous processes according to an embodiment of the present invention.

Furthermore, FIG. 8 is a perspective view showing an overall structure of the locker of the spacer unit, that is, a principal part of the spacer device for fixing a band between spinous processes according to an embodiment of the present invention.

It may be seen that the present invention has a structure in which a spacer unit (300) is fixed to a band unit (200) as shown.

The band unit (200) has a specific degree of tension. The band unit (200) is disposed between a spinous process (501p) and adjacent spinous process (502p) protruded from a vertebra (501) and in the outskirts of the spinous process (501p) and the outskirts of the adjacent spinous process (502p), and fixes the spinous process (501p) and the adjacent spinous process (502p).

For reference, hereinafter, reference numeral 501p is used for a spinous process protruded from the vertebra (501). Such a spinous process is named a first spinous process (501p), for convenience sake.

Furthermore, reference numeral 502p is used for a spinous process adjacent to the first spinous process (501p). Such a spinous process is named a second spinous process (502p), for convenience sake, in order to distinguish it from the first spinous process (501p).

Furthermore, reference numeral 501 is used for a vertebra from which the first spinous process (501p) has protruded. Such a vertebra is named a first vertebra (501), for convenience sake.

Furthermore, reference numeral 502 is used for a vertebra from which the first spinous process (502p) has protruded. Such a vertebra is named a second vertebra (502), for convenience sake, in order to distinguish it from the first vertebra 501.

The spacer unit (300) is coupled to the band unit (200) and disposed in the space between the first spinous process (501p) and the second spinous process (502p), and fixes the end of the band unit (200).

In this case, the spacer unit (300) moves from the outside of the body of a patient to the inside of the body along the band unit (200) in the state in which the band unit (200) has penetrated the spacer unit (300), and is inserted into the space between the first spinous process (501p) and the second spinous process (502p).

That is, as will be described later in more detail, the spacer unit (300) moves along the band unit (200) in the direction in which the band unit (200), which is disposed between the first spinous process (501p) and the second spinous process (502p) and in the outskirts of the first spinous process (501p) and the outskirts of the second spinous process (502p) and is extended to fix the first spinous process (501p) and the second spinous process (502p) as described above, is formed and is then inserted into the inside of the body of the patient.

Accordingly, according to the present invention, upon a procedure for treating a vertebral disease, the end of the band unit (200) which fixes the first and the second spinous processes (501p and 502p) adjacent to each other, but permits a minute movement can be certainly fixed by the spacer unit (300). Furthermore, a minimal invasive surgery (MIS) is made possible because the body has only to be incised to the extent that the spacer unit (300) moves along the band unit (200) and moves into the body.

Furthermore, the present invention has a relatively cheap and simple configuration including a spacer body (310), a tightner (320), and a locker (330) along with the band unit 200. Accordingly, a procedure can be efficiently performed regardless of a skill, and optimal procedure results can be provided to a patient.

Furthermore, the present invention can handle various body types of patients because the band unit (200) is closely attached and fixed to the first spinous process 501p and the second spinous process (502p) while permitting a gap between the first spinous process (501p) and the second spinous process (502p) to some extent.

Accordingly, according to the present invention, the state in which the spacer unit (300) fixes the end of the band unit (200) is maintained. Accordingly, a patient can be provided with the same state as that of the original vertebra because a fast fixing state is maintained and a minute movement is permitted after a procedure.

The above embodiment may be applied to the present invention and the following various embodiments may also be applied to the present invention.

First, it may be seen that the spacer unit (300) has a structure including the spacer body (310) and the tightner (320).

A main band (210) that belongs to the band unit (200) and that is disposed between the first spinous process (501p) and the second spinous process (502p) penetrates the spacer body (310).

The tightner (320) is coupled to the spacer body (310), and the main band (210) penetrates the tightner (320).

Accordingly, an assistant band (220) coupled to the main band (210) penetrates the tightner (320) via the outskirts of the first spinous process (501p). The other end of the main band (210) penetrates the tightner (320) via the outskirts of the second spinous process (502p).

Furthermore, it may be seen that each of the main band (210) and the assistant band (220) is fixed through the tightner (320).

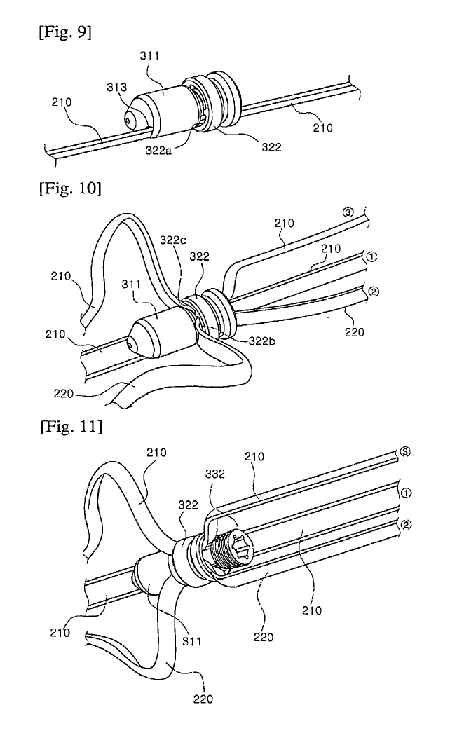

Furthermore, as will be described later, the present invention may provide convenience in a procedure because the main band (210) and the assistant band (220) penetrate the spacer body (310) in the state in which the tightner (320) has been coupled to the spacer body (310), as shown in FIGS. 9 to 11.

Meanwhile, the spacer unit (300) may further include the locker (330) coupled to the tightner (320) in order to fix the ends of the main band (210) and the assistant band (220).

Furthermore, the spacer unit (300) may additionally include a clamp (340) which is fixed to the outer surface of the tightner (320) and finishes the fixed ends of the main band (210) and the assistant band (220) through the tightner (320).

Meanwhile, an embodiment having a structure, including the main band (210) having a first length and permitting elastic transform and the assistant band (220) having a second length shorter than the first length, permitting elastic transform, and having one end coupled at a specific point of the main band (210), may be applied to the band unit (200).

Accordingly, the other end \textcircled{2} of the assistant band (220) is disposed in the outskirts of the first spinous process (501p), one end \textcircled{3} of the main band (210) is disposed in the outskirts of the second spinous process (502p), and the other end \textcircled{1} of the main band (210) is disposed after passing through the space between the first spinous process (501p) and the second spinous process (502p).

For reference, one end of the assistant band (220) has been fixed to the main band (210) by a clamp piece (230, hereinafter refer to FIG. 1).

That is, the other end \textcircled{1} of the main band (210) penetrates the spacer unit (300).

The spacer body (310) is described in more detail with reference to FIG. 6. It may be seen that the spacer body (310) includes a main body (311), a first screw thread (312), and a band slot (313).

The main body (311) is a member of a barrel shape having one side open. The first screw thread (312) is depressed and formed from the other end of the main body (311) to one end and is screwed onto the tightner (320).

The band slot (313) is penetrated from one end of the main body (311) to the other end on one side of the main body (311) so that the other end \textcircled{1} of the main band (210) is inserted into the band slot (313) from the one end side of the main body (311) and thus the other end \textcircled{1} of the main band (210) is exposed from the other end side of the main body (311).

In this case, the main body (311) is not limited to a special shape, but it is preferred that the main body (311) has a cylindrical shape so that it is easily inserted into the body of a patient upon a procedure.

In this case, it is preferred that the spacer body (310) further includes a peak part (314) formed from one end of the main body (311) and narrowed toward the front end so that the spacer body (310) is easily inserted into the body of a patient.

The tightner (320) is described in more detail with reference to FIG. 7. It may be seen that the tightner (320) has a structure including a first fastening part (321), a penetration part (322), and a second fastening part (323).

A second screw thread (321t) is formed in the outer surface of the first fastening part (321), and thus the first fastening part (321) is screwed onto the spacer body (310).

The penetration part (322) is provided at the end of the first fastening part (321). A first communication slot (322a) through which the other end \textcircled{1} of the main band (210) penetrating the spacer body (310) penetrates, a second communication slot (322b) through which the other end \textcircled{2} of the assistant band (220) penetrates, and a third communication slot (322c) through which one end \textcircled{3} of the main band (210) penetrates penetrate the penetration part (322) in the direction in which they are fastened to the spacer body (310). The first communication slot (322a), the second communication slot (322b), and the third communication slot (322c) are disposed in penetration part (322) at equal intervals.

The second fastening part (323) is depressed from the end of the penetration part (322) toward the first fastening part (321). A third screw thread (323t) is formed in the inner surface of the second fastening part (323).

In this case, a hook groove (324) of a ring shape is depressed in the outer surface of the penetration part (322). The clamp (340) is hung and fixed to the hook groove 324.

Hook pieces (342) hung and fixed to the hook groove (324) are spaced apart at equal intervals and disposed in the clamp (340). The hook pieces (342) finish the ends of main band (210) and the assistant band (220) fixed through the tightner (320).

The locker (330) is described in more detail with reference to FIG. 8. It may be seen that the locker (330) has a structure including a third fastening unit (331) and a fixing body (332).

That is, the locker (330) is screwed onto the third screw thread (323t) of the second fastening part (323) formed in the penetration part (322), and fixes the other end \textcircled{1} and one end of the main band (210) and the end of the assistant band (220).

Accordingly, the first communication slot (322a), the second communication slot (322b), and the third communication slot (322c) communicate with the inside of the penetration part (322), that is, a receiving groove (322g).

A fourth screw thread (331t) screwed onto the third screw thread (323t) is formed in the outer surface of the third fastening unit (331).

The fixing body (332) is provided at the end of the third fastening unit (331) and stepwise formed from the end of the penetration part (322) toward the second fastening part (323). The fixing body (332) is seated and fixed to the receiving groove (322g) communicating with the first communication slot (322a), the second communication slot (322b), and the third communication slot (322c), and fixes the main band (210) and the assistant band (220).

Accordingly, the main band (210) and the assistant band (220) are fixed between the inner surface of the receiving groove (322g) and the outer surface of the fixing body (332).

Meanwhile, the locker (330) further includes a tool groove (333) which is depressed from the end of the fixing body (332) toward the third fastening unit (331) and which corresponds to a shape of the end of a separately provided fastening tool (hereinafter not shown), thereby being capable of promoting convenience in assembly and fastening.

Furthermore, it is preferred that the locker (330) further includes a fixed pattern (334) which forms mountains and valleys in a ring shape along the outer surface of the fixing body (332) so that the state in which the ends (\textcircled{1}, \textcircled{2}, and \textcircled{3}) of each of the main band (210) and the assistant band (220) have been closely attached and fixed can be certainly maintained.

It may be seen that the fixing body (332) is gradually widened from the third fastening unit (331) toward the end side on which the tool groove (333) has been formed.

A process for sequentially coupling the spacer unit to the band unit using the spacer device for fixing a band between spinous processes according to an embodiment of the present invention and performing a procedure is described with reference to FIGS. 9 to 11.

For reference, reference is made to FIGS. 1 to 8, for reference numeral not indicated in FIGS. 9 to 11.

First, a procedure person penetrates the other end \textcircled{1} of the main band (210) through the band slot (313) and the first communication slot (332a) in the state in which the spacer body (310) and the tightner (320) have been coupled as shown in FIG. 9.

In this case, disposing the spacer body (310) between the first spinous process (501p) and the second spinous process (502p) is the same as that shown in FIG. 1 and described above.

Thereafter, the procedure person penetrates one end \textcircled{3} of the main band (210) through the third communication slot (332c) while penetrating the other end \textcircled{2} of the assistant band (220) through the second communication slot (332b) as shown in FIG. 10, and couples the locker (330) to the penetration part (322) of the tightner (320) as shown in FIG. 11, thereby completing a specific step of the procedure.

As described above, it may be seen that the present invention has its basic technical spirit to provide the spacer device for fixing a band between the spinous processes, wherein upon a procedure for treating a vertebral disease, the end of the band which fixes adjacent spinous process, but permits a minute movement can be fixed efficiently and certainly, a procedure can be efficiently performed regardless of a skill and optimal procedure results can be obtained through a relatively cheap and simple configuration, an action can be taken in accordance with various body types of patients, and a patient can be provided with the same state as that of the original vertebra by permitting a minute movement while maintaining a fast fixing state after a procedure.

Furthermore, many other modifications and applications are also possible by a person having ordinary skill in the art without departing from the basic technical spirit of the present invention.

### DESCRIPTION OF REFERENCE NUMERALS OF PRINCIPAL ELEMENTS IN THE DRAWINGS

200 . . . band unit
210 . . . main band
220 . . . assistant band
230 . . . clamp piece
300 . . . spacer unit
310 . . . spacer body
311 . . . main body
312 . . . first screw thread
313 . . . band slot
314 . . . peak part
320 . . . tightner
321 . . . first fastening part
321t . . . second screw thread
322 . . . penetration part
322a . . . first communication slot
322b . . . second communication slot
322c . . . third communication slot
322g . . . receiving groove
323 . . . second fastening part
323t . . . third screw thread
324 . . . hook groove
330 . . . locker
331 . . . third fastening unit
331t . . . fourth screw thread
332 . . . fixing body
333 . . . tool groove
334 . . . fixed pattern
340 . . . clamp
342 . . . hook pieces
501 . . . first vertebra (vertebra)
502 . . . second vertebra (adjacent vertebra)
501p . . . first spinous process (spinous process)
502p . . . second spinous process (adjacent spinous process).

What is claimed is:

1. A spacer device for fixing a band between spinous processes, comprising:
a band unit having a specific degree of tension, being disposed between a spinous process and adjacent spinous process protruded from vertebras and in outskirts of the spinous process and outskirts of the adjacent spinous process, and fixing the spinous process and the adjacent spinous process; and
a spacer unit coupled to the band unit, disposed in a space between the spinous process and the adjacent spinous process, and having an end of the band unit,
wherein the spacer unit moves from an outside of a body to an inside of the body along the band unit in the state in which the spacer unit has penetrated the band unit and is inserted into the space between the spinous process and the adjacent spinous process.

2. The spacer device of claim 1, wherein:
the spacer unit comprises a spacer body through which a main band belonging to the band unit and disposed between the spinous process and the adjacent spinous process penetrates and a tightner which is coupled to the spacer body and through which the main band penetrates,
an assistant band coupled to the main band penetrates the tightner via the outskirts of the spinous process,
the other end of the main band penetrates the tightner via the outskirts of the adjacent spinous process, and
each of the main band and the assistant band is fixed through the tightner.

3. The spacer device of claim 2, wherein the main band and the assistant band penetrate the tightner in the state in which the tightner has been coupled to the spacer body.

4. The spacer device of claim 2, wherein the spacer unit further comprises a locker which is coupled to the tightner and which fixes ends of the main band and the assistant band.

5. The spacer device of claim 2, wherein the spacer unit further comprises a clamp which is fixed to an outside surface of the tightner and which finishes ends of the main band and the assistant band fixed through the tightner.

6. The spacer device of claim 1, wherein:
the band unit comprises a main band having a first length and permitting elastic transform and an assistant band having a second length shorter than the first length, permitting elastic transform, and having one end coupled at a specific point of the main band,
the other end of the assistant band is disposed in the outskirts of the spinous process,
one end of the main band is disposed in the outskirts of the adjacent spinous process,
the other end of the main band is disposed after passing through the space between the spinous process and the adjacent spinous process, and
the other end of the main band penetrates the spacer unit.

7. The spacer device of claim 2, wherein the spacer body comprises:
a main body of a barrel shape having one side open,
a first screw thread depressed and formed from the other end of the main body toward one end and screwed onto the tightner, and
a band slot penetrated from one end of the main body to the other end on one side of the main body so that the other end of the main band is inserted into the band slot from one end side of the main body and the other end of the main band is exposed from the other end side of the main body.

8. The spacer device of claim 2, wherein the tightner comprises:
a first fastening part screwed onto the spacer body, a second screw thread being formed in an outer surface of the fast fastening part,
a penetration part provided at an end of the first fastening part, wherein a first communication slot through which the other end of the main band penetrating the spacer body penetrates, a second communication slot through which an end of the assistant band penetrates, and a third communication slot through which one end of the main band penetrates penetrate the penetration part in a direction in which the first to third communication slots are fastened to the spacer body, the first communication slot, the second communication slot, and the third communication slot being disposed in the penetration part at equal intervals, and
a second fastening part depressed from an end of the penetration part toward the first fastening part, a third screw thread being formed in an inner surface of the second fastening part.

9. The spacer device of claim 8, further comprising a locker which is screwed onto the third screw thread of the second fastening part formed in the penetration part and which fixes the other end and one end of the main band and the end of the assistant band,
wherein the first communication slot, the second communication slot, and the third communication slot communicate with an inside of the penetration part.

10. The spacer device of claim 9, wherein:
the locker comprises a third fastening unit which has a fourth screw thread screwed onto the third screw thread formed in an outer surface of the third fastening unit and a fixing body which is provided at an end of the third fastening unit, stepwise formed from the end of the penetration part toward the second fastening part, and seated and fixed to a receiving groove communicating with the first communication slot, the second communication slot, and the third communication slot and which fixes the main band and the assistant band, and
the main band and the assistant band are fixed between an inner surface of the receiving groove and an outer surface of the fixing body.
